(19) United States
(12) Patent Application Publication
(10) Pub. No.: US 2014/0371095 A1
(43) Pub. Date: Dec. 18, 2014

(54) NOVEL METHOD FOR IDENTIFYING SPECIFIC MARKER SEQUENCES FOR PROSTATE CANCER

(71) Applicant: Protagen AG, Dortmund (DE)

(72) Inventors: Peter Amersdorfer, Graz (AT); Angelika Lueking, Bochum (DE); Axel Kowald, Bochum (DE); Bettina Schlick, Innsbruck (AT); Petra Massoner, Innsbruck (AT); Christof Seifart, Innsbruck (AT); Georg Schäfer, Innsbruck (AT); Helmut Klocker, Inzing (AT); Peter Schulz-Knappe, Hemmingen (DE); Klaus Marquart, Dortmund (DE)

(73) Assignee: Protagen AG, Dortmund (DE)

(21) Appl. No.: 14/357,806

(22) PCT Filed: Nov. 14, 2012
(86) PCT No.: PCT/EP2012/072658

§ 371 Date: Aug. 18, 2014

(30) Foreign Application Priority Data
Nov. 14, 2011 (EP) 11189067.9

(51) Int. Cl. G01N 33/574

(52) U.S. Cl. CPC G01N 33/57434
USPC 506/9; 506/18; 506/17

(57) ABSTRACT

The present invention relates to a novel method for identifying specific marker sequences for diagnosis of prostate cancer and/or for prognosis in prostate cancer, and to the use of the specific marker sequences identified with the aid of this method.

The present invention relates to a novel method for identifying specific marker sequences for diagnosis of prostate cancer and/or for prognosis in prostate cancer and also to the use of the identified specific marker sequences.

Protein biochips are gaining increasing industrial importance in analysis and diagnosis as well as in pharmaceutical development. Protein biochips have become established as screening tools.

Here, the rapid and highly parallel detection of a multiplicity of specifically binding analysis molecules in a single experiment is made possible. To produce protein biochips, it is necessary to have the required proteins available. In particular, protein expression libraries have been established for this purpose. High-throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is closely linked to the progress of the genome sequencing projects and the annotation of these gene sequences. In addition, the determination of the expressed sequence is not always clear due to differential splicing processes. This problem can be avoided by the use of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). Here, the cDNA of a specific tissue is cloned into a bacterial or eukaryotic expression vector, such as yeast. The vectors used for the expression are generally characterised in that they carry inducible promoters that may be used to control the time of protein expression. In addition, expression vectors have sequences for what are known as affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

By way of example, the gene products of a cDNA expression library from human foetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from expression libraries could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Such protein biochips based on cDNA expression libraries are disclosed in particular in WO 99/57311 and WO 99/57312.

Furthermore, in addition to antigen-presenting protein biochips, antibody-presenting arrays are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

The prevalence, incidence and mortality rate of prostate cancer are rising globally. Prostate cancer is the second most common fatal cancer among men. However, the incidence of prostate carcinoma here is much higher than the mortality: not every patient suffering from prostate cancer develops a progressive form. Progression of the prostate carcinoma is observed in only approximately one quarter of all cases, but leads to an aggressive, metastasising form of the disease, which results in death and for which there previously have been no efficient therapy options (Jemal, A., et al., Global cancer statistics. CA Cancer J Clin, 2011. 61(2): p. 69-90.). Biomarkers that are hardly validated are currently described, which allow a differentiation and prediction of the course of progressive/aggressive and non-progressive form of prostate cancer.

Various studies indicate that prostate cancer and inflammation are related to one another (Dennis, L. K., C. F. Lynch, and J. C. Torner, Epidemiologic association between prostatitis and prostate cancer. Urology, 2002. 60(1): p. 78-83; Sarma, A. V., et al., Sexual behaviour, sexually transmitted diseases and prostatitis: the risk of prostate cancer in black men. J Urol, 2006. 176(3): p. 1108-13). It is supposed that inflammatory processes in the prostate tissue (prostatitis) are accompanied by a high number of infiltrating immune cells and distinct cytokine and chemokine profile (De Marzo, A. M., et al., Inflammation in prostate carcinogenesis. Nat Rev Cancer, 2007. 7(4): p. 256-69; Culig, Z., Cytokine disbalance in common human cancers. Biochim Biophys Acta, 2011. 1813(2): p. 308-14; Maitland, N. J. and A. T. Collins, Inflammation as the primary aetiological agent of human prostate cancer: a stem cell connection? J Cell Biochem, 2008. 105(4): p. 931-9; Robert G., et al., Biomarkers for the diagnosis of prostatic inflammation in benign prostate hyperplasia. The Prostate 2011, 71: 1709-1711). Prostatitis is therefore a possible risk factor for prostate cancer which may also influence the progression of the disease. Prostatitis is currently diagnosed exclusively by means of biopsy and histopathological analysis.

WO2010/000874, in the name of the applicant, for example describes the diagnosis of prostate carcinoma and prostate inflammation by means of a protein biochip and provides certain diagnostic marker sequences for prostate cancer. Here, it was made possible for the first time to sensitively identify these marker sequences for the respective indications by means of protein biochips.

Certain genes and expression products thereof are associated with chronic inflammatory processes of the prostate. Robert et al. (The Prostate 2011, 71, 1701-1709) discloses the fact that CCR4, CCR7, CD40LG, CTLA4, ICOS, IL17, PTPRC, SELP and TFRC are significantly associated with chronic inflammatory processes in the prostate. Barbieri et al. (Histopathology 2012, 60, 187-198) describes non-synonymous point mutations in the SPOP gene which are associated with prostate cancer.

However, there is also a significant need to improve the diagnosis of prostate cancer and prognosis in prostate cancer. Here, there is a need in particular for markers for prostate cancer which allow simple and quick diagnosis and also prognosis.

The invention relates to a method for identifying specific marker sequences for diagnosis of prostate cancer and/or for the prognosis for prostate cancer, comprising the following steps:

a.) Selecting patients with prostate cancer and high inflammation values and/or patients with prostate cancer and low inflammation values,

b.) Determining the interaction of a sample from the selected patients with marker sequences to be tested, wherein the marker sequences to be tested are placed on a solid support,

c.) Selecting marker sequences that demonstrate an interaction, and

d.) Determining whether the selected marker sequences differ between progressive and non-progressive prostate cancer (specific marker sequences).

Chronic prostate inflammation is one of the main causes for false-positive serum PSA values during the routine health check of men from 50 years of age. The latest tests indicate that an inflamed microenvironment in the prostate promotes the development of the malignancy and progression of the metastatic disease. In spite of these findings, the diagnosis of chronic prostatitis is still possible only by means of a biopsy. This places stress both on the patient and on the health system. With the aid of the method according to the invention, simple non-invasive biomarkers (specific marker sequences) can be identified, which can be used as an indicator for inflammation of the prostate, preferably chronic inflammation and the early development or diagnosis of prostate cancer, preferably prostate carcinoma.

The provision of specific marker sequences permits a reliable diagnosis, prognosis and stratification of patients suffering from disorders ranging from prostate inflammatory diseases to prostate carcinoma. In a particularly preferred embodiment of the invention, the marker sequences to be tested are localised on a solid support. The marker sequences to be tested are particularly preferably used (presented) in the method according to the invention by means of a protein biochip.

With the development of prostate cancer, an immune response is produced in the body, since the patients produce antibodies against cancer cells, whereby prostate cancer-specific autoantibodies and autoantibody profiles are formed. These autoantibodies and autoantibody profiles can be detected in samples taken from the patient/test subject.

The cellular changes during the course of the development from inflammatory processes in the prostate to prostate cancer, for example the formation of antigens, in particular prostate cancer-specific antigens, are identified by the immune system as “foreign”. Autoantibodies are then produced in a target-oriented and specific manner by B cells. Both the antigens and autoantibodies, which can be formed during the course of the prostate cancer development and the progression of prostate cancer, may be specific marker sequences that can be identified using the method according to the invention. The antigens formed in conjunction with the inflammation or an inflammatory process in the prostate/in the prostate tissue and also the autoantibodies formed subsequently are inflammation markers in the sense of this invention. The invention relates to the identification and use of these inflammation markers.

The specific marker sequences, for example inflammation markers, allow a differentiation of the progressive/aggressive and non-progressive forms of prostate cancer as well as a prognostic test for prediction of the course of a prostate disease or prostate cancer. One embodiment of the invention concerns a method for identifying specific marker sequences, wherein the selected marker sequences, with low inflammation values, differ between progressive and non-progressive prostate cancer. In a particularly preferred embodiment of the method according to the invention, the selected marker sequences differ between progressive and non-progressive prostate cancer with high inflammation values.

In one embodiment of the invention, the marker sequences to be tested are the selected sequences, the specific marker sequences and inflammation markers and/or autoantigens and/or autoantibodies.

A particularly preferred embodiment of the invention concerns methods for identifying specific marker sequences, wherein it is determined by means of histological methods whether the selected marker sequences differ between progressive (malignant) and non-progressive (benign) prostate cancer, for example by means of the immunohistochemistry on prostate tissue.

The invention enables the identification of antigens and autoantigens as specific marker sequences for the inflammatory component in prostate cancer or prostate cancer development and/or progression. The invention also enables a delimitation of prostate cancer, in particular aggressive/progressive prostate cancer and benign inflammatory processes, such as prostatitis and BPH (benign prostate hyperplasia). The invention therefore also relates to a correlation of inflammatory reactions in the prostate with antibodies and/or autoantibodies, for example in the serum.

In a preferred embodiment of the method according to the invention, the selected patients or test subjects belong to the same population group (population).

The method can be applied to various populations and may lead there to the identification of different specific marker sequences. A main embodiment of the invention therefore concerns methods for identifying specific marker sequences, wherein the selected patients belong to one population. A population is constituted by organisms that belong to a certain species, preferably homo sapiens, and live in a certain geographical area. Examples of populations are “Europeans, Americans, Asians”. In the sense of the invention, a population may also mean a conformity in terms of certain genetic parameters, for example a conformity with respect to a genetic predisposition for prostate cancer and/or for inflammation parameters and/or inflammation markers. The method according to the invention is therefore also suitable for applications within the scope of personalised medicine.

In one embodiment of the method according to the invention, the sample of the selected patients or test subjects is a bodily fluid or a tissue sample, in particular blood, whole blood, blood plasma, blood serum, patient serum, urine, cerebrospinal fluid, or synovial fluid.

Inflammation-specific autoantibodies for example from blood serum of prostate cancer patients can be detected for example in comprehensive expression libraries.

A further embodiment of the invention concerns methods for identifying specific marker sequences, wherein the marker sequences are mRNA, si-RNA, microRNA, cDNA, peptide or protein, in particular antigens or autoantigens, or originate from an expression library, in particular an mRNA, si-RNA, microRNA, cDNA, peptide or protein expression library.

Patient samples can be divided into highly inflamed and mildly inflamed test samples, for example on the basis of the number of tissue-infiltrating lymphocytes. The corresponding serum samples can be analysed on protein microarrays. These protein microarrays preferably consist of more than 3,000 to 5,000 cancer- and inflammation-associated (recombinant) proteins. The comparison of the autoantibody profiles of patient groups with highly inflamed and mildly inflamed tissue results in the determination of specific marker sequences for prostate cancer.

In total, 997 different autoantibodies were detected in both patient groups (highly inflamed and mildly inflamed tissue). Significantly higher values for the individual autoantibodies were observed for 176 antigens with severe inflammation in the prostate carcinoma. The calculation of the diagnostic potential of this screening study gave a sensitivity value of 64% and a specificity value of 65%. These specific marker sequences have a diagnostic selectivity of 0.71.

In one embodiment of the method according to the invention, the marker sequences to be tested are selected from the group comprising SEQ ID No. 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

In one embodiment of the method according to the invention, the marker sequences to be tested are presented on a protein microarray.

The invention also relates to the use of one or more specific marker sequences obtainable by a method according to the invention for diagnosis of prostate cancer, preferably for diagnosis of prostate carcinoma.

The invention also relates to the use of one or more specific marker sequences obtainable by a method according to the invention for prognosis in prostate cancer and/or for stratification, in particular for risk stratification, or for therapy monitoring in prostate cancer.

The invention also relates to the use of SPOP and/or partial sequences and/or homologues of SPOP for diagnosis of prostate cancer and/or for prognosis in prostate cancer and/or for stratification in prostate cancer. SPOP (Homo sapiens speckle-type POZ protein) has the GI accession number gi/56117827.

The invention also relates to the use of STX18 and/or partial sequences and/or homologues of STX18 for diagnosis of prostate cancer and/or for prognosis in prostate cancer and/or for stratification in prostate cancer. STX18 (Homo sapiens syntaxin 18) has the GI accession number gi/39725935.

The invention also relates to the use of SPAST and/or partial sequences and/or homologues of SPAST for diagnosis of prostate cancer and/or for prognosis in prostate cancer and/or for stratification in prostate cancer. SPAST (Homo sapiens spastin) has the GI accession number gi/40806168.

Within the scope of studies forming the basis of the invention, three autoantibodies with significantly higher sensitivity and specificity values in the serum samples from the highly inflamed patients were firstly selected and characterised in greater detail. For all other marker sequences identified within the scope of this invention (SEQ ID No. 1 to 528), this characterisation can be applied similarly. It was possible to show by means of immunohistochemistry that SPAST, STX18 and SPOP are present in the epithelium of benign (non-progressive prostate cancer) and malignant (progressive prostate cancer) prostate areas. All three marker proteins demonstrate a much higher colour intensity in tissue sections from the highly inflamed patient groups. Here, interestingly, the highest colour intensity was able to be determined in tissue-infiltrating lymphocytes.

The invention therefore also relates to the use of the specific marker sequences obtainable using the method according to the invention, in particular the specific marker sequences SEQ ID N. 1 to 528, for example of SPOP, STX18 and/or SPAST, to distinguish between benign prostate cancer and malignant prostate cancer.

The invention also relates to an arrangement of specific marker sequences obtainable by a method according to the invention for diagnosis of prostate cancer and/or prognosis in prostate cancer and/or for stratification in prostate cancer. Here, the arrangement according to the invention may comprise or consist of one or more specific marker sequences obtainable by the method according to the invention.

The invention also relates to an arrangement of specific marker sequences obtainable by a method according to the invention for diagnosis of prostate cancer and/or prognosis in prostate cancer and/or for stratification in prostate cancer comprising or consisting of one or more specific marker sequences, and wherein the specific marker sequences are selected from the group comprising SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

The invention also relates to an arrangement of specific marker sequences obtainable by a method according to the invention comprising or consisting of SPOP and/or partial sequences of SPOP and/or homologues of SPOP and/or STX18 and/or partial sequences of STX18 and/or homologues of STX18 and/or SPAST and/or partial sequences of SPAST and/or homologues of SPAST. The invention also relates to an arrangement according to the invention of specific marker sequences for diagnosis of prostate cancer and/or prognosis in prostate cancer and/or stratification in prostate cancer.

The invention also relates to an assay or protein microarray comprising an arrangement according to the invention of specific marker sequences and optionally further additives and excipients. The invention also relates to an assay or protein microarray (protein biochip) comprising an arrangement of specific marker sequences on a solid support.

The invention also relates to the use of an arrangement according to the invention or of an assay according to the invention or of a protein microarray according to the invention for identifying and characterising a substance for prostate cancer, in particular prostate carcinoma-containing agent for the detection of binding success, wherein a.) the arrangement or the assay or the protein microarray is brought into contact with at least one substance to be tested, and b.) binding success is detected.

The invention also relates to a diagnostic agent for diagnosis of prostate cancer and/or prognosis in prostate cancer, comprising an arrangement according to the invention and/or one or more specific marker sequences obtainable by a method according to the invention and/or selected from the group comprising specific marker sequences SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528, in particular SPOP and/or STX18 and/or SPAST and/or a partial sequence and/or a homologue sequence thereof.

The invention also relates to a kit for diagnosis or prognosis or stratification of prostate cancer diseases containing one or more specific marker sequences obtainable by a method according to the invention and/or one or more of the marker sequences selected from the group comprising SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues SEQ ID No. 1-528 with at least 90%, preferably at least 95%, of the length of SEQ ID No. 1-528, for example SPOP and/or STX18 and/or SPAST and/or a partial sequence and/or a homologue sequence.

The protein sequences SEQ ID No. 1-176, the DNA clone sequences (SEQ ID No. 177-352 and the RNA sequences SEQ ID No. 253-528 are specified in the accompanying sequence protocol, which forms part of this application.

The invention also relates to the use of a specific marker sequence obtained by a method according to the invention or selected from one of the sequences SEQ ID No. 1-528 or from SPOP or STX18 or SPAST or a partial sequence or a homologue sequence as affinity material for performing apheresis or blood washing in patients with prostate cancer.

The invention also relates to a target for the treatment and therapy of prostate cancer obtained by a method according to the invention or selected from one of the sequences SEQ ID No. 1-528 or from SPOP or STX18 or SPAST or a partial sequence or a homologue sequence.

The invention also relates to the use of an arrangement according to the invention or of an assay according to the invention for the screening of active agents (substances) for prostate cancer, in particular prostate carcinoma.

The invention also relates to a method for diagnosis of prostate cancer or for prognosis in prostate cancer, wherein

a.) one or more specific marker sequences obtained by a method according to the invention and/or one or more the selected marker sequences SEQ ID. No. 1-528 and/or SPOP and/or STX18 and/or SPAST and/or a partial sequence and/or a homologue sequence is/are applied to a solid support and

b.) is/are brought into contact with the bodily fluid or the tissue sample from a test subject or patient, and

c.) an interaction of the bodily fluid or tissue sample with the marker sequences from a.) is detected.

A particular embodiment of the invention concerns methods for the early detection and diagnosis of prostate cancer, wherein the interaction according to c.) indicates a prostate cancer-associated autoantibody profile of the patient or of a cohort or of a population group (population) or of a specific disease progression (prognosis) or of a certain response to a therapy/drug.

One or more specific marker sequences is/are used in a diagnosis method and/or in a diagnostic agent, a protein microarray or an arrangement. In a preferred embodiment, at least 2, for example 3, 4, 5, 6, 7, 8, 9, 10, preferably 15 to 20 marker sequences or 30 to 50 or 100 or more specific marker sequences are used together or in combination, for example directly in succession or in parallel.

An interaction of the bodily fluid or of the tissue sample with the specific marker sequence or marker sequences can be detected for example by means of a probe, in particular by means of an antibody.

The prediction of progression and/or an early diagnosis can make it possible, in the case of prostate cancer or threat of prostate cancer, for the patient in question to be treated and/or monitored in good time if it is determined that a severe progression of the disease is likely. In such a case, the patient can be closely monitored and/or treated in good time. On the other hand, patients can be identified in which a mild form of the disease and/or spontaneous healing is/are likely. In such a case, it may be that no treatment is necessary, which could contribute to savings in the health service.

The invention concerns embodiments in which 2 or more specific marker sequences, for example 3, 4, or 5 or more, 10 to or more, preferably 30 to 50 marker sequences or 50 to 100 or more marker sequences are determined on a patient to be tested.

In a further embodiment of the invention, the specific marker sequences according to the invention can also be supplemented or expanded with known biomarkers for this indication.

The stratification of patients with prostate inflammatory diseases up to prostate carcinoma in new or established sub-groups of prostate inflammation diseases up to prostate carcinoma, and the appropriate selection of patient groups for the clinical development of new therapeutic substances is also included. The term “therapy control” also includes the allocation of patients to responders and non-responders regarding a therapy or the therapy course thereof.

In the sense of this invention, “diagnosis” means the positive determination of prostate inflammatory diseases up to prostate carcinoma by means of the marker sequences according to the invention as well as the assignment of the patients to the prostate inflammatory disease up to prostate carcinoma. The term diagnosis includes the medical diagnostics and examinations in this regard, in particular in-vitro diagnostics and laboratory diagnostics, and also proteomics and nucleic acid blotting. Further tests may be necessary to be sure and to exclude other diseases. The term diagnosis therefore also includes the differential diagnosis of prostate inflammatory diseases and prostate carcinoma by means of the marker sequences according to the invention, and the prognosis of the prostate inflammatory diseases or prostate carcinoma.

In the sense of this invention, “stratification or therapy control” means that the method according to the invention renders possible decisions for the treatment and therapy of the patient, whether it is the hospitalisation of the patient, the use, efficacy and/or dosage of one or more drugs, or the monitoring of the course of a disease and the course of therapy or aetiology or classification of a disease, for example into a new or existing sub-type, or the differentiation of diseases and patients thereof.

In a further embodiment of the invention, the term “stratification” in particular includes the risk stratification with the prognosis of an “outcome” of a negative health event.

Within the scope of this invention, the term “patient” is understood to mean any test subject (human or mammal), with the provision that the test subject is tested for prostate inflammatory diseases to prostate carcinoma.

The terms “prostate inflammatory diseases”, “prostate cancer”, “prostate carcinoma” comprise a group of diseases from prostatitis to the chronic forms of all prostate inflammations and the establishment thereof as prostate cancer or prostate carcinoma (Definition for example according to Pschyrembel, de Gruyter, 261. Edition (2007), Berlin).

Prostate cancer includes all cancer diseases of the prostate, in particular prostate carcinoma. Prostate cancer includes all forms of the disease, that is to say progressive/aggressive forms and non-progressive forms.

“Prostate cancer-specific” or “specific” means that the marker sequence, for example the nucleic acid or the polypeptide or protein obtainable therefrom, interacts with substances from the bodily fluid or tissue sample from a patient with prostate cancer (for example antigen (epitope)/antibody (paratope) interaction). These substances from the bodily fluid or tissue sample either only occur or are expressed or occur or are expressed at least in an intensified manner in the case of prostate cancer, whereas these substances in patients without prostate cancer are not present or are only present to a smaller extent (smaller quantity, lower concentration). On the other hand, specific marker sequences can also be characterised in that they interact with substances from the bodily fluid or tissue sample from patients with prostate cancer because these substances no longer occur or are no longer expressed or only occur or are expressed at least in a much lower quantity/concentration in the case of prostate cancer, whereas these substances are present in patients without prostate cancer or are at least present to a much greater extent. Specific marker sequences may also be present in healthy test subjects, however the quantity (concentration) thereof changes for example with the development, establishment and therapy of prostate cancer. The specific marker sequences are therefore biomarkers for prostate cancer. The specific marker sequences may thus indicate a profile of substances from bodily fluid and tissue sampling, for example a prostate cancer-associated autoantibody profile.

“Prostate cancer-associated autoantibody profiles” thus include on the one hand the composition (one or more autoantibodies) and on the other hand the quantity/concentration of individual autoantibodies.

In a particularly preferred embodiment of the invention, the specific marker sequence is an antigen or part of an antigen or codes for an antigen or for part of an antigen.

In a particularly preferred embodiment, the specific marker sequence identifies/binds to autoantibodies that are present (intensified) during the course of the development, establishment and therapy of prostate cancer or are present to a smaller extent (or are no longer present). Autoantibodies are formed by the body against the body's own antigens, which for example are produced when prostate cancer is present. Autoantibodies are formed by the body against different substances and pathogens. Within the scope of the present invention, the autoantibodies in particular that are formed with the occurrence of and during the course of the development of prostate cancer and/or of which the expression is upregulated or downregulated are detected. These autoantibodies can be detected with the aid of the method according to the invention and specific marker sequences and are therefore used as an indication for prostate cancer. The detection and the monitoring of the quantity of prostate cancer-associated autoantibodies in the patient can be used for the early detection, diagnosis and/or therapy monitoring/therapy control. These autoantibody profiles may be sufficiently characterised already with use of a specific marker sequence. In other cases, two or more specific marker sequences are necessary in order to indicate a prostate cancer-associated autoantibody profile.

In preferred embodiments of the invention, these autoantibodies can be detected using specific marker sequences, which are derived from another individual, because they originate for example from a commercial cDNA bank.

In other preferred embodiments of the invention, these autoantibodies can be detected using specific marker sequences, which are derived from the same individual (autoantigen) because they originate for example from a cDNA bank produced especially for the patient or a group of patients (for example within the scope of personalised medicine. By way of example, homologues of the aforementioned specific marker sequences are then used, for example sequences that have non-synonymous mutations in the specific marker sequences.

Autoantibodies can be formed by the patient already many years before the occurrence of the first symptoms of the disease. Early detection, diagnosis and also prognosis and (preventative) treatment would therefore be possible years before the visible outbreak of the disease. The devices and means (arrangement, array, protein biochip, diagnostic agent, test kit) and methods according to the invention thus enable a very early intervention compared with known methods, which considerably improves the prognosis and survival rates. Since the prostate cancer-associated autoantibody profiles change during the establishment and treatment/therapy of prostate cancer, the invention also enables the detection and the monitoring of prostate cancer at any stage of development and treatment and also monitoring within the scope of aftercare. The means according to the invention also allow easy handling at home by the patient and cost-effective routine precautionary measures for early detection.

In particular due to the use of antigens as specific marker sequence for prostate cancer, which derive from sequences already known, for example from commercial cDNA banks, test subjects can be tested, and, where applicable, prostate cancer-associated autoantibodies present in these test subjects can be detected, even if the corresponding autoantigens are not (yet) known in this test subject.

Different patients may have different prostate cancer-associated autoantibody profiles, for example different cohorts or population groups (populations) differ from one another. Here, each patient may form one or more different prostate cancer-associated autoantibodies during the course of the development of prostate cancer and the progression of the disease, that is to say also different autoantibody profiles. In addition, the composition and/or the quantity of the formed specific autoantibodies may change during the course of the development and progression of the disease, such that a quantitative evaluation is necessary. The therapy/treatment of prostate cancer also leads to changes in the composition and/or the quantity of prostate cancer-associated autoantibodies. The large selection of specific marker sequences according to the invention allows the individual compilation of specific marker sequences in an arrangement for individual patients, groups of patients, certain cohorts, population groups, etc. In an individual case, the use of a specific marker sequence may therefore be sufficient, whereas in other cases at least two or more specific marker sequences have to be used together or in combination in order to produce a meaningful autoantibody profile.

Compared with other biomarkers, the detection of autoantibodies for example in the serum/plasma has the advantage of high stability and storage capability and good detectability. The presence of autoantibodies also is not subject to a circadian rhythm, and therefore the sampling is independent of the time of day, food intake and the like.

In addition, the prostate cancer-associated autoantibodies can be detected with the aid of the corresponding antigens/autoantigens in known assays, such as ELISA or Western Blot, and the results can be checked for this.

The term “specific marker sequence” in the sense of this invention means that the cDNA or the polypeptide or protein obtainable therefrom is significant for prostate inflammatory diseases and/or prostate cancer, for example prostate carcinoma. By way of example, the cDNA or the polypeptide or protein obtainable therefrom may interact with substances from the bodily fluid or tissue sample from a patient with prostate inflammatory diseases up to prostate carcinoma (for example antigen (epitope)/antibody (paratope) interaction).

It is essential to the invention that an interaction between the bodily fluid or tissue sample from a patient and the specific marker sequences is detected. Such an interaction is, for example, a bond, in particular a binding substance on at least one marker sequence according to the invention or in the case of a cDNA the hybridisation with a suitable substance under selected conditions, in particular stringent conditions (for example as defined conventionally in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology”, Green Publishing Associates and Wiley Interscience, N.Y. (1989)). One example of stringent hybridisation conditions is: hybridisation in 4×SSC at 65° C. (alternatively in 50% formamide and 4×SSC at 42° C.), followed by a number of washing steps in 0.1×SSC at 65° C. for a total of approximately one hour. An example of less stringent hybridisation conditions is hybridisation in 4×SSC at 37° C., followed by a number of washing steps in 1×SSC at room temperature.

The marker sequences to be tested and/or the specific marker sequences, in a further embodiment of the invention, have a recognition signal that is addressed to the substance to be bound (for example antibody, nucleic acid). In accordance with the invention, the recognition signal for a protein is preferably an epitope and/or paratope and/or hapten, and for a cDNA is preferably a hybridisation or binding region.

Examples of specific marker sequences that are obtainable in accordance with the method according to the invention are detailed in Tables 1 and 2 and can also be clearly identified by the respectively cited database entry (also accessible by Internet: http://www.ncbi.nlm.nih.gov/) (see in Table 1 (annex) and Table 2: accession no.), see also the associated sequence protocol.

The invention therefore also concerns the full-length sequences of the marker sequences according to the invention, more specifically as defined in Table 1 via the known database entry according to Table 1.

In accordance with the invention, the marker sequences also comprise modifications of the cDNA sequence and the corresponding amino acid sequence, such as chemical modification, such as citrullination, acetylation, phosphorylation, glycosylation or polyA strand and further modifications known as appropriate to a person skilled in the art.

In a further embodiment of the invention partial sequences (partial sequences also include fragments) or homologues of the marker sequences according to the invention are also included. Marker sequences according to the invention in the sense of the invention are specific marker sequences, marker sequences having SEQ ID No. 1-528 in accordance with the accompanying sequence protocol, SPOP, STX18 and SPAST.

The invention also includes the full-length sequences of the specific marker sequences SEQ ID No. 1-528 according to the invention.

Furthermore, embodiments of SEQ 1-528 analogue to (homologues, partial sequences of) the specific marker sequences SEQ 1-528, as presented for example in the claims, are therefore also included, since the SEQ 1-528 according to the invention in turn represent partial sequences, at least with high homology. However, the specific marker sequences SEQ 1-528 are preferred in accordance with the invention.

The invention also relates to homologues of the specific marker sequences and partial sequences, for example fragments of specific marker sequences.

For example, homologues are nucleic acid sequences and/or protein sequences that have an identity with the specific marker sequences of at least 70% or 80%, preferably 90% or 95%, particularly preferably 96% or 97% or more, for example 98% or 99% or more. In a particularly preferred embodiment of the invention, for the case in which the specific marker sequences are antigens, the homology in the sequence range in which the antigen-antibody or anti-autoantibody interaction takes place, is at least 95%, preferably at least 97%, particularly preferably at least 99%. In accordance with the invention, homologues produced by mutations such as base exchange mutations, raster mutations, base insertion mutations, base loss mutations, point mutations, or insertion mutations, for example, are included.

The invention also relates to partial sequences of the specific marker sequences. Partial sequences also include fragments of the marker sequences according to the invention, and partial sequences are nucleic acids or proteins/peptides that are shortened compared with the entire nucleic acid or the entire protein/peptide. Here, the deletion may occur at the end or the ends and/or within the sequence. For example, partial sequences and/or fragments that have 50 to 100 nucleotides, 70-120 nucleotides of an entire sequence are included, for example of SEQ 1-528. Homologues of partial sequences and fragments are also included in accordance with the invention. In a particular embodiment, the specific marker sequences are shortened compared with the sequences 1-528 to such an extent that they still consist only of the binding point(s) for the prostate cancer-associated autoantibody in question. In accordance with the invention, specific marker sequences are also included that differ from the sequences SEQ ID No. 1-528 in that they contain one or more insertions, wherein the insertions for example are 1 to 100 or more nucleotide/amino acids long, preferably 5 to 50, particularly preferably 10 to 20 nucleotides/amino acids long and the sequences are otherwise identical however or homologous to sequences 1-1578. Partial sequences that have at least 90%, preferably at least 95%, particularly preferably at least 97% or 98%, of the length of the specific marker sequences according to the invention are particularly preferred. In accordance with the invention, homologues of the partial sequences are also included. Homologues of the specific marker sequences that have one or more non-synonymous point mutations are particularly preferred.

The marker protein SPOP for example displays mutations of the SPOP gene. The described mutations were detected in 6-13% of the prostate cancer cases and lead to the loss of the protein activity. Since the production of prostate cancer associated antigens is caused inter alia by mutations, a total of 25 tissue samples were subjected to RNA sequencing within the scope of this invention. 23 samples had the wildtype sequence, however the mutation of the 134 codon was discovered in two of the samples. This SPOP mutation is a possible reason for the observed immunogenicity/immune response. In particular, non-synonymous point mutations in specific marker sequences SEQ ID No. 1 to 528 and the homologues deriving therefrom of the specific marker sequences SEQ ID No. 1 to 528 are therefore included in accordance with the invention.

Homologues or partial sequences of the marker sequences are in particular also of interest for the early detection, diagnosis, prognosis and therapy control in individual patient groups/population groups within the scope of personalised medicine, since the specific markers in question in the serum etc., for example the prostate cancer-specific autoantibodies, may differ from patient group/population group to patient group/population group.

In a further embodiment, the respective specific marker sequence can be represented in different quantities in one or more regions on the support. This allows a variation of the sensitivity. The regions may each have a totality of specific marker sequences, that is to say a sufficient number of different specific marker sequences, in particular 2, 3, 4, 5, 6, 7, 8, 9 or 10 or more different specific marker sequences, and where applicable further nucleic acids and/or proteins, in particular biomarkers.

In a further embodiment, the respective marker sequence can be represented in different quantities in one or more regions on a solid support. This permits a variation of the sensitivity. The regions may each have a totality of marker sequences, that is to say a sufficient number of different marker sequences, in particular 2 to 5 or 10 or more and optionally more nucleic acids and/or proteins, in particular biomarkers. However, at least 96 to 25,000 (numerical) or more from different or identical marker sequences and further nucleic acids and/or proteins, in particular biomarkers, are preferred. Furthermore preferred are more than 2,500, particularly preferably 10,000 or more different or identical marker sequence and optionally further nucleic acids and/or proteins, in particular biomarkers.

The invention also relates to arrangements of marker proteins. The arrangement preferably contains at least 2 to 5 or 10, preferably 30 to 50 marker sequences, or 50 to 100 or more marker sequences.

Within the scope of this invention, “arrangement” is synonymous with “array”, and, if this “array” is used to identify substances on marker sequences, this is to be understood to be an “assay” or a diagnostic device. In a preferred embodiment, the arrangement is designed such that the marker sequences represented on the arrangement are present in the form of a grid on a solid support. Furthermore, those arrangements are preferred that permit a high-density arrangement of protein binders and the marker sequences are spotted. Such high-density spotted arrangements are disclosed for example in WO 99/57311 and WO 99/57312 and can be used advantageously in a robot-supported automated high-throughput method.

Within the scope of this invention, however, the term “assay” or diagnostic device likewise comprises those embodiments of a device such as ELISA, bead-based assay, line assay, Western Blot, and immunochromatographic methods (for example what are known as lateral flow immunoassays) or similar immunological single or multiplex detection methods. A protein biochip in the sense of this invention is the systematic arrangement of proteins on a solid support.

The marker sequences of the arrangement are fixed on a solid support, but are preferably spotted or immobilised or even printed on, that is to say applied in a reproducible manner. One or more marker sequences can be present multiple times in the totality of all marker sequences and may be present in different quantities based on a spot. Furthermore, the marker sequences can be standardised on the solid support (for example by means of serial dilution series of, for example, human globulins as internal calibrators for data normalisation and quantitate evaluation).

The invention therefore concerns an assay or protein biochip consisting of an arrangement containing marker sequences according to the invention.

In a further embodiment, the marker sequences are present as clones. Such clones can be obtained for example by means of a cDNA expression library according to the invention (Büssow et al. 1998 (above)). In a preferred embodiment, such expression libraries containing clones are obtained using expression vectors from a cDNA expression library consisting of the cDNA marker sequences. These expression vectors preferably contain inducible promoters. The induction of the expression can be carried out for example by means of an inducer, such as IPTG. Suitable expression vectors are described in Terpe et al. (Terpe T Appl Microbiol Biotechnol. 2003 January; 60(5):523-33).

Expression libraries are known to a person skilled in the art; they can be produced in accordance with standard works, such as Sambrook et al, “Molecular Cloning, A laboratory handbook, 2nd edition (1989), CSH press, Cold Spring Harbor, N.Y. Expression libraries that are tissue-specific (for example human tissue, in particular human organs) are furthermore preferable. Further, expression libraries that can be obtained by means of exon-trapping are also included in accordance with the invention. Instead of the term expression library, reference may also be made synonymously to an expression bank.

Protein biochips or corresponding expression libraries that have do not exhibit any redundancy (what is known as a Uniclone® library) and that can be produced in accordance with the teaching of WO 99/57311 and WO 99/57312 are furthermore preferred. These preferred Uniclone® libraries have a high proportion of non-defective fully expressed proteins of a cDNA expression library.

Within the scope of this invention, the clones can also be, but are not limited to, transformed bacteria, recombinant phages or transformed cells of mammals, insects, fungi, yeasts or plants.

The clones are fixed, spotted or immobilised on a solid support.

The invention therefore relates to an arrangement, wherein the marker sequences are present as clones.

In addition, the marker sequences can be present in the respective form of a fusion protein, which for example contains at least one affinity epitope or “tag”. The tag may be or may contain one such as c-myc, his tag, arg tag, FLAG, alkaline phosphatase, V5 tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

In all embodiments, the term “solid support” includes embodiments such as a filter, a membrane, a magnetic or fluorophore-labelled bead, silicon wafer, glass, metal, plastic, a chip, a mass spectrometry target or a matrix. However, a filter is preferred in accordance with the invention.

Furthermore, PVDF, nitrocellulose or nylon is preferred as a filter (for example Immobilon P Millipore, Protran Whatman, Hybond N+Amersham).

In a further preferred embodiment of the arrangement according to the invention, this corresponds to a grid with the dimensions of a microtiter plate (8-12 well strips, 96 wells, 384 wells or more), a silicon wafer, a chip, a mass spectrometry target or a matrix.

A substance to be tested may be any native or non-native biomolecule, a synthetic chemical molecule, a mixture or a substance library.

Once the substance to be tested contacts the marker sequence, the binding success is evaluated, and is performed for example with use of commercially available image analysing software (GenePix Pro (Axon Laboratories), Aida (Raytest), ScanArray (Packard Bioscience).

Interactions, for example protein-protein interactions (for example protein on the marker sequence to be tested or specific marker sequence, such as antigen/antibody) or corresponding “means for detecting the binding success” can be visualised for example by means of fluorescence labelling, biotinylation, radio-isotope labelling or colloid gold or latex particle labelling in the conventional manner. For example, bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemoluminescent substrates. A readout is performed for example by means of a microarray laser scanner, a CCD camera or visually.

The following FIGURES and examples explain the invention, but do not limit the invention to the examples.

FIG. 1: volcano plot illustration of the 176 marker sequences (see Example 2).

### EXAMPLE 1

### Pre-Selection of the Patient Cohorts

The examined patients were divided into a group with low inflammation values and a group with high inflammation values and were characterised in terms of age size of the carcinoma, Gleason score, C-reactive protein, volume of the prostate, weight of the prostate, PSA and fPSA %. Prostate tissue of 70 patients with prostate cancer was examined histologically. Here, tissue samples were dyed immunohistochemically with p63 (blue) and CD45 (brown). The evaluation of the staining is intended primarily to clarify the question of how many immune cells are present in tumour areas compared with benign prostate areas. The difference between tumour/benign prostate can be ascertained on the basis of the p63 staining (benign with p63 positive).

An overview of the inflammation state in prostate tissues in cancer patients was made possible by means of IHC double staining. An ideal staining for an evaluation with HistoFaxs (Tissuegnostics) constitutes a p63 staining for distinction between tumour/benign tissue in red (FastRed) and a CD45 staining for detection of leucocytes in brown (DAP). Infiltrating immune cells can be detected by the pan-leucocyte marker CD45 in prostate tissue. As a result of this colour combination, p63 and CD45 are clearly evident compared with the haematoxylin counterstaining. The evaluation in the HistoFAXS was performed with the aid of the “single reference shade” (HistoQuant Program, Tissuegnostics).

Cytotoxic T-lymphocytes can be detected by the surface marker CD8 in prostate tissues. The counterstaining (tissue identification) is performed using haematoxylin.

32 patients with prostate cancer and low inflammation values and 38 patients with prostate cancer and high inflammation values were identified in this way. All patients belonged to a certain population.

### EXAMPLE 2

### Identification of Specific Marker Sequences (Autoantibody Screening)

For the screening in a pilot test, a protein microarray from Protagen AG was used, which, in addition to ˜2,000 randomly selected human antigens, contains more than 1,500 further autoantigens, which were identified in prior internal tests in the indications prostate cancer, breast cancer, systemic lupus erythematosus (SLE), multiple sclerosis, rheumatoid arthritis and juvenile idiopathic arthritis. The sera of 32 patients with prostate cancer and low inflammation values and the sera of 38 patients with prostate cancer and high inflammation values were examined on this microarray. Specific markers for high and for low inflammation were determined via different bioinformatic/biostatistical approaches, such as single marker ranking by means of Mann Whitney Test, Volcano Plot analysis and classifications by means of support vector machines (SVM).

In this test, 997 different autoantibodies were identified in both patient cohorts (low inflammation, high inflammation) and in all serum samples. From this, the 176 specific marker sequences were determined in the highly inflamed serum samples (p<0.05; Mann-Whitney test; fold change >2).

In this test, 176 specific marker sequences were identified (see FIG. 1 volcano plot illustration). The amino acid sequences, the corresponding DNA sequences and the corresponding RNA sequences are specified in the accompanying sequence protocol under SEQ ID No. 1-528. In Table 1 (annex), the naming of the 176 protein sequences and also the accession number under which the proteins are stored are specified.

Table 2 reproduces a selection of 30 protein sequences that, interestingly, had already been identified as marker sequences in the case of prostate cancer during the course of this test (see WO2010/000874).

TABLE 2

| | | | p- | Fold |
|---|---|---|---|---|
| Rank | Name | GI Accession | Value | Change |
| 1 | Homo sapiens spastin (SPAST); transcript variant 1; mRNA | gi\|40806168 | 0.001 | 14.33 |
| 2 | Homo sapiens ribosomal protein L36a-like (RPL36AL); mRNA | gi\|34335143 | 0.001 | 5.16 |
| 5 | FEZ family zinc finger 2 | gi\|157388917 | 0.002 | 0.16 |
| 6 | Homo sapiens makorin, ring finger protein, 1 (MKRN1), mRNA | gi\|21359891 | 0.002 | 11.19 |
| 7 | acyl-CoA thioesterase 7 isoform hBACHd | gi\|32528286 | 0.003 | 6.11 |
| 15 | Homo sapiens ubiquitin-conjugating enzyme E2O (UBE2O); mRNA | gi\|33636749 | 0.005 | 11.81 |
| 21 | Homo sapiens coactosin-like (Dictyostelium) (COTL1), mRNA | gi\|23510452 | 0.007 | 7.44 |
| 22 | Homo sapiens tetratricopeptide repeat domain 5 (TTC5); mRNA | gi\|24308431 | 0.007 | 2.97 |
| 24 | IKBE_HUMAN NF-kappaB inhibitor epsilon (NF-kappa-BIE) (I-kappa-B-epsilon) | gi\|14548073 | 0.007 | 3.59 |
| | (IkappaBepsilon) (IKB-epsilon) (IKBE) | | | |
| 25 | Homo sapiens famesyl diphosphate synthase (famesyl pyrophosphate synthetase; | gi\|41281370 | 0.008 | 15.80 |
| | dimethylallyltranstransferase; geranyltranstransferase) (FDPS); mRNA | | | |
| 26 | Homo sapiens cyclin-dependent kinase 7 (MO15 homolog; Xenopus laevis; cdk-activating | gi\|16950659 | 0.008 | 22.02 |
| | kinase) (CDK7); mRNA | | | |
| 27 | low density lipoprotein receptor-related protein associated protein 1 | gi\|4505021 | 0.008 | 3.37 |
| 28 | Homo sapiens surfeit 5 (SURF5); transcript variant c; mRNA | gi\|31652217 | 0.008 | 9.33 |
| 32 | Homo sapiens tubulin, alpha 3 (TUBA3), mRNA | gi\|17986282 | 0.008 | 13.30 |
| 34 | Homo sapiens G protein-coupled receptor 161 (GPR161); transcript variant 2; mRNA | gi\|24476015 | 0.009 | 4.12 |
| 35 | Homo sapiens chromosome 2 genomic contig; alternate assembly (based on Celera assembly) | gi\|88958353 | 0.009 | 5.48 |
| 36 | Homo sapiens NOL1/NOP2/Sun domain family; membsr 5 (NSUN5); transcript variant 2; mRNA | gi\|23199996 | 0.009 | 3.03 |
| 37 | Homo sapiens chromosome 9 genomic contig. reference assembly | gi\|89029256 | 0.010 | 3.84 |
| 39 | similar to homoprotocatechuate catabolism bifunctional isomerase/decarboxylase | gi\|14336767 | 0.010 | 2.57 |
| 40 | PREDICTED: Homo sapiens similar to Myc-associated zinc finger protein (MAZI) (Purine- | gi\|113426244 | 0.011 | 5.00 |
| | binding transcription factor) (Pur-1) (ZF87) (ZIF87) (LOC642773), mRNA | | | |
| 41 | Homo sapiens chromosome 9 genomic contig. reference assembly | gi\|89029256 | 0.011 | 9.40 |
| 49 | Homo sapiens upstream binding transcription factor. RNA polymerase I (UBTF), mRNA | gi\|7657670 | 0.012 | 10.36 |
| 50 | Homo sapiens APC11 anaphase promoting complex subunit 11 homolog (yeast) (ANAPC11), | gi\|50409803 | 0.012 | 21.41 |
| | transcript variant 4, mRNA | | | |
| 51 | ribosomal protein L35a | gi\|16117791 | 0.012 | 16.39 |
| 54 | myotonic dystrophy protein kinase isoform 1 | gi\|126091095 | 0.014 | 3.37 |
| 55 | Homo sapiens hypothetical protein FLJ12949 (FLJ12949). transaipt variant 2, mRNA | gi\|30410780 | 0.014 | 3.92 |
| 56 | Homo sapiens nerve growth factor receptor (TNFRSF16) associated protein 1 (NGFRAP1); | gi\|46094059 | 0.014 | 11.31 |
| | transcript variant 2; mRNA | | | |
| 58 | Homo sapiens FERM; RhoGEF and pleckstrin domain protein 2 (FARP2); mRNA | gi\|7662309 | 0.014 | 4.65 |
| 59 | Homo sapiens chromosome 11 genomic contig; alternate assembly (based on Celera | gi\|89034479 | 0.014 | 4.64 |
| | assembly) | | | |
| 60 | Homo sapiens brain expressed X-linked 2 (BEX2) mRNA | gi\|50658085 | 0.014 | 4.02 |

### EXAMPLE 3

### Validation of the Specific Marker Sequences on Tissue Cuts/FFPE

In order to provide a sensible validation of the autoantibodies in accordance with a screen (in conjunction with the biostatistical evaluation), a testing of individual selected marker sequences (here, four selected marker proteins from Table 1) on FFPE tissue cuts from the prostate of patients with prostate cancer was performed in this step by means of IHC analyses. All other marker sequences SEQ ID No. 1-528 have to be characterised similarly in order to identify herefrom the marker sequences specific for the certain population.

The prostate tissue of 3 patients was tested.

The marker sequences (here marker proteins (=autoantigens)) were selected in accordance with the following principles—a) availability of IHC reagents, that is to say primary antibodies available for purchase, b) relevance of the marker from the literature, such as TTLL12, which plays a part in prostate tumour genesis, and c) the human protein atlas, where the further markers SPAST, SPOP, STX19 have already been analysed immunohistochemically. The result was successful for the IHC analysis.

TABLE 3
Primary antibodies available for purchase for IHC analysis

| Primary | | | | |
|---|---|---|---|---|
| Antibody | company | dilution | group | |
| SPAST | Sigma | 1:50 | High | Low |
| | HPA017311 | | inflammation, | inflammation, |
| | | | high AAB in | no AAB in |
| | | | serum | serum |
| SPOP | Sigma | 1:50 | High | Low |
| | SAB1406659 | | inflammation, | inflammation, |
| | | | high AAB in | no AAB in |
| | | | serum | serum |
| STX19 | Sigma | 1:100 | High | Low |
| | HPA003019 | | inflammation, | inflammation, |
| | | | high AAB in | no AAB in |
| | | | serum | serum |
| TTLL12 | | 1:100 | High | Low |
| | | | inflammation, | inflammation, |
| | | | high AAB in | no AAB in |
| | | | serum | serum |

It was possible to demonstrate that lymphocyte-infiltrated prostrate tissue displayed greater stain reactions for all four markers. SPOP and STX18 demonstrate a differentiated staining and are thus suitable for distinction between benign and cancer in areas in the prostate with low inflammation.

SPOP (speckle-type POZ protein) modulates the transcriptional repression activity of the death-associated protein 6 (DAXX): E3 ubiquitin ligase gene. SPOP plays a part in TNF-conveyed JNK signalling (kidney cancer). SPOP is mutated in prostate cancer. SPOP is localised in the nucleus.

### Immunohistochemistry (ICH):

With greater inflammation, SPOP demonstrates high autoantibody staining by means of IHC in prostate cells, wherein the staining in tissue cuts with prostate cancer is greater than in benign prostate cells. With processes demonstrating less inflammation, SPOP does not demonstrate autoantibody staining in prostate cells (Be greater than Ca).

### STX18 (Syntaxin 18)

Stx18 is a Q-SNARE (soluble N-ethylmaleimide-sensitive factor attachment receptor) protein associated with the endoplasmatic reticulum. Stx18 is a cell growth-inhibiting gene 9 protein. Implications in breast cancer and a moderate expression in prostate cancer are described for Stx18. Stx18 stains the plasma membrane in ICC.

### Immunohistochemistry:

With greater inflammation, Stx18 demonstrates a deep staining (high autoantibody mirror), but no difference between benign cells and cancer cells. With processes demonstrating less inflammation, Stx19 does not demonstrate autoantibody staining in prostate cells (Be greater than Ca).

SPAST (Spastin) belongs to the protein family that divides an ATPase domain and plays a part in various cellular processes, for example in membrane transport, intracellular mobility, biogenesis of organelles, protein folding and proteolysis. ATPase may be involved with the formation/arrangement or function of the nucleoprotein complex. SPAST stains the cytoplasm in prostate cells moderately and stains the membranes positively.

### Immunohistochemistry:

With greater inflammation, SPAST demonstrates deep staining (high autoantibody mirror), but no difference between benign cells and cancer cells. With processes demonstrating less inflammation, SPAST demonstrates no autoantibody staining in prostate cells and no difference between benign cells and cancer cells.

TTLL12 (tubulin tyrosine ligase-like family, member 12) (no AAB) reads (converts) tyrosine to a-tubulin, which is terminally detyrosinated. TTLL12 is important for neuronal organisation, trafficking of intermediate filament proteins, cell morphology and spindle positioning. TTLL12 expression is supressed during tumour progression (increase in detyrosinated tubulin), and the aggressiveness of the tumour increases. TTLL12 is expressed in the proliferating layer of benign prostate cells. The expression of TTLL12 increases with progressive cancer to metastasis. In many cell lines which are derived from metastasising prostate cancer cells the expression of TTLL12 is highly regulated. A downregulation of the TTLL12 expression has an effect on different post-translational modifications of tubulin. An over-expression of TTLL12 changes the chromosome ploidy.

### Immunohistochemistry:

With greater inflammation, TTLL12 demonstrates deep staining (high autoantibody mirror), wherein benign cells demonstrate a much greater staining than cancer cells. With processes demonstrating less inflammation, TTLL12 demonstrates no autoantibody staining in prostate cells and a very heterogeneous staining of the prostate cells.

All tested primary antibodies detect autoantibodies in prostate cells. They demonstrate a much deeper staining in highly inflammatory processes than in mildly inflammatory processes.

The targets of three autoantibodies that were found in blood serum samples of test subjects with highly inflammatory prostate cancer are expressed in prostate tissue.

Lymphocytes which infiltrate the prostate tissue demonstrated a deeper staining than the prostate tissue itself. This effect was found with all four tested primary antibodies.

STX18 and SPOP demonstrated different quantities in areas with prostate cancer and areas with benign epithelial cells. STX18 and SPOP were able to distinguish between benign and prostate cancer areas with mildly inflammatory prostate cancer. For further analysis, areas with mildly inflammatory tissue were tested with and without autoantibodies in blood serum samples.

Highly inflammatory areas are characterised by a high concentration of infiltrating lymphocytes. Mildly inflammatory areas are characterised by low or no infiltration of lymphocytes.

The results show that, with highly inflammatory processes, the four tested selected marker sequences do not demonstrate any differences in the staining with progressive/aggressive and non-invasive prostate cancer. In the case of mildly inflammatory processes in the prostate, the selected marker sequences SPOP and STX118, however, demonstrate a significant difference in the staining with progressive/aggressive prostate cancer compared with non-invasive prostate cancer. SPOP and STX18 (both demonstrate a deeper staining with non-invasive/benign prostate cancer) are therefore specific marker sequences in the sense of the invention. They are suitable for diagnosis, prognosis and stratification.

### EXAMPLE 4

### Point Mutations in SPOP

The RNA was extracted and sequenced from 25 tissue samples from patients with high inflammation values. The mutation of the K134R in the tissue samples of 2 patients was detected in SPOP (Mutation in Codon 134).

TABLE 1

| SEQ | | | | |
|---|---|---|---|---|
| ID | | | Protein | |
| No. | Gene ID | Taxonomy | Accession No | Definition |
| 1 | 7541 | 9606 | gi\|19923242 | zinc finger protein 161 homologue [Homo sapiens] |
| 2 | 54617 | 9606 | gi\|38708321 | INO80 complex homologue 1 [Homo sapiens] |
| 3 | 29889 | 9606 | gi\|7019419 | guanine nucleotide binding protein-like 2 (nucleolar) [Homo sapiens] |
| 4 | 25776 | 9606 | gi\|7656942 | PKD2 interactor, golgi and endoplasmic reticulum associated 1 isoform a [Homo sapiens] |
| 5 | 11135 | 9606 | gi\|23238226 | CDC42 effector protein 1 [Homo sapiens] |
| 6 | 7358 | 9606 | gi\|4507813 | UDP-glucose dehydrogenase [Homo sapiens] |
| 7 | 79656 | 9606 | gi\|187828564 | BEN domain containing 5 [Homo sapiens] |
| 8 | 9925 | 9606 | gi\|7662074 | zinc finger and BTB domain containing 5 [Homo sapiens] |
| 9 | 6166 | 9606 | gi\|4506651 | ribosomal protein L36a-like protein [Homo sapiens] |
| 10 | 5878 | 9606 | gi\|41393545 | RAB5C, member RAS oncogene family isoform b [Homo sapiens] |
| 11 | 4843 | 9606 | gi\|24041029 | nitric oxide synthase 2A [Homo sapiens] |
| 12 | 9126 | 9606 | gi\|4885399 | structural maintenance of chromosomes 3 [Homo sapiens] |
| 13 | 1844 | 9606 | gi\|4758206 | dual specificity phosphatase 2 [Homo sapiens] |
| 14 | 439 | 9606 | gi\|50428938 | arsA arsenite transporter, ATP-binding, homologue 1 [Homo sapiens] |
| 15 | 203 | 9606 | gi\|4502011 | adenylate kinase 1 [Homo sapiens] |
| 16 | 4794 | 9606 | gi\|71274109 | nuclear factor of kappa light polypeptide gene enhancer in B-cells inhibitor, epsilon [Homo sapiens] |
| 17 | 10274 | 9606 | gi\|62243696 | stromal antigen 1 [Homo sapiens] |
| 18 | 2098 | 9606 | gi\|33413400 | S-formylglutathione hydrolase [Homo sapiens] |
| 19 | 88 | 9606 | gi\|4501893 | actinin, alpha 2 [Homo sapiens] |
| 20 | 59343 | 9606 | gi\|54607091 | SUMO1/sentrin/SMT3 specific protease 2 [Homo sapiens] |
| 21 | 91875 | 9606 | gi\|226498382 | tetratricopeptide repeat protein 5 [Homo sapiens] |
| 22 | 7419 | 9606 | gi\|25188179 | voltage-dependent anion channel 3 isoform b [Homo sapiens] |
| 23 | 8424 | 9606 | gi\|4502369 | gamma-butyrobetaine dioxygenase [Homo sapiens] |
| 24 | 54934 | 9606 | gi\|154426300 | hypothetical protein LOC54934 [Homo sapiens] |
| 25 | 7073 | 9606 | gi\|4507499 | TIA-1 related protein isoform 1 [Homo sapiens] |
| 26 | 9170 | 9606 | gi\|7305013 | lysophosphatidic acid receptor 2 [Homo sapiens] |
| 27 | 51440 | 9606 | gi\|7705419 | hippocalcin-like protein 4 [Homo sapiens] |
| 28 | 644096 | 9606 | gi\|111038124 | hypothetical protein LOC644096 [Homo sapiens] |
| 29 | 57645 | 9606 | gi\|22027480 | pogo transposable element with KRAB domain [Homo sapiens] |
| 30 | 975 | 9606 | gi\|4757944 | CD81 antigen [Homo sapiens] |
| 31 | 2954 | 9606 | gi\|22202624 | glutathione transferase zeta 1 isoform 1 [Homo sapiens] |
| 32 | 10765 | 9606 | gi\|57242796 | jumonji, AT rich interactive domain 1B [Homo sapiens] |
| 33 | 81889 | 9606 | gi\|215422413 | fumarylacetoacetate hydrolase domain containing 1 isoform 3 [Homo sapiens] |
| 34 | 23450 | 9606 | gi\|54112121 | splicing factor 3b, subunit 3 [Homo sapiens] |
| 35 | 6117 | 9606 | gi\|4506583 | replication protein A1, 70 kDa [Homo sapiens] |
| 36 | 3033 | 9606 | gi\|94557308 | L-3-hydroxyacyl-Coenzyme A dehydrogenase precursor [Homo sapiens] |
| 37 | 1663 | 9606 | gi\|100913204 | probable ATP-dependent RNA helicase DDX11 isoform 2 [Homo sapiens] |
| 38 | 1022 | 9606 | gi\|4502743 | cyclin-dependent kinase 7 [Homo sapiens] |
| 39 | 112812 | 9606 | gi\|72534754 | ferredoxin 1-like [Homo sapiens] |
| 40 | 716 | 9606 | gi\|4502495 | complement component 1, s subcomponent [Homo sapiens] |
| 41 | 8882 | 9606 | gi\|4508021 | zinc finger protein 259 [Homo sapiens] |
| 42 | 1760 | 9606 | gi\|126091095 | myotonic dystrophy protein kinase isoform 1 [Homo sapiens] |
| 43 | 337867 | 9606 | gi\|221316645 | RecName: Full = Ubiquitin-associated domain-containing protein 2; AltName: Full = Phosphoglycerate |
| | | | | dehydrogenase-like protein 1; Flags: Precursor |
| 44 | 9770 | 9606 | gi\|7661964 | Ras association domain family 2 [Homo sapiens] |
| 45 | 4000 | 9606 | gi\|5031875 | lamin A/C isoform 2 [Homo sapiens] |
| 46 | 55816 | 9606 | gi\|29544726 | docking protein 5 [Homo sapiens] |
| 47 | 22902 | 9606 | gi\|7662352 | RUN and FYVE domain containing 3 isoform 2 [Homo sapiens] |
| 48 | 9570 | 9606 | gi\|16905522 | golgi SNAP receptor complex member 2 isoform A [Homo sapiens] |
| 49 | 58491 | 9606 | gi\|11034821 | zinc finger protein 71 [Homo sapiens] |
| 50 | 55746 | 9606 | gi\|26051235 | nucleoporin 133 kDa [Homo sapiens] |
| 51 | 39 | 9606 | gi\|148539872 | acetyl-Coenzyme A acetyltransferase 2 [Homo sapiens] |
| 52 | 22934 | 9606 | gi\|94536842 | ribose 5-phosphate isomerase A (ribose 5-phosphate epimerase) [Homo sapiens] |
| 53 | 7170 | 9606 | gi\|114155146 | tropomyosin 3 isoform 3 [Homo sapiens] |
| 54 | 9726 | 9606 | gi\|215820619 | zinc finger protein 646 [Homo sapiens] |
| 55 | 89953 | 9606 | gi\|41871946 | kinesin-like 8 isoform a [Homo sapiens] |
| 56 | 88745 | 9606 | gi\|24308350 | hypothetical protein LOC88745 [Homo sapiens] |
| 57 | 64221 | 9606 | gi\|48476182 | roundabout, axon guidance receptor, homologue 3 [Homo sapiens] |
| 58 | 4594 | 9606 | gi\|156105689 | methylmalonyl Coenzyme A mutase precursor [Homo sapiens] |
| 59 | 23020 | 9606 | gi\|40217847 | activating signal cointegrator 1 complex subunit 3-like 1 [Homo sapiens] |
| 60 | 51287 | 9606 | gi\|46198304 | coiled-coil-helix-coiled-coil-helix domain containing 8 [Homo sapiens] |
| 61 | 51308 | 9606 | gi\|42476206 | receptor accessory protein 2 [Homo sapiens] |
| 62 | 7343 | 9606 | gi\|7657671 | upstream binding transcription factor, RNA polymerase I isoform a [Homo sapiens] |
| 63 | 6434 | 9606 | gi\|4759098 | splicing factor, arginine/serine-rich 10 [Homo sapiens] |
| 64 | 22920 | 9606 | gi\|18105054 | kinesin-associated protein 3 [Homo sapiens] |
| 65 | 83939 | 9606 | gi\|54873624 | eukaryotic translation initiation factor 2A [Homo sapiens] |
| 66 | 79363 | 9606 | gi\|241666479 | RecName: Full = Miro domain-containing protein C1orf89 |
| 67 | 476 | 9606 | gi\|21361181 | Na+/K+-ATPase alpha 1 subunit isoform a proprotein [Homo sapiens] |
| 68 | 27018 | 9606 | gi\|7657044 | nerve growth factor receptor (TNFRSF16) associated protein 1 isoform b [Homo sapiens] |
| 69 | 79891 | 9606 | gi\|190610006 | zinc finger protein 671 [Homo sapiens] |
| 70 | 57687 | 9606 | gi\|24308257 | vesicle amine transport protein 1 homologue (T. californica)-like [Homo sapiens] |
| 71 | 60528 | 9606 | gi\|145553959 | elaC homologue 2 [Homo sapiens] |
| 72 | 23608 | 9606 | gi\|223468622 | makorin, ring finger protein, 1, isoform CRA_e [Homo sapiens] |
| 73 | 3550 | 9606 | gi\|125988409 | RED protein [Homo sapiens] |
| 74 | 11188 | 9606 | gi\|66472382 | nischarin [Homo sapiens] |
| 75 | 4665 | 9606 | gi\|5174607 | NGFI-A binding protein 2 [Homo sapiens] |
| 76 | 5442 | 9606 | gi\|110618253 | mitochondrial DNA-directed RNA polymerase precursor [Homo sapiens] |
| 77 | 23246 | 9606 | gi\|21327667 | block of proliferation 1 [Homo sapiens] |
| 78 | 90326 | 9606 | gi\|42734379 | THAP domain containing, apoptosis associated protein 3 [Homo sapiens] |
| 79 | 3104 | 9606 | gi\|4885419 | zinc finger and BTB domain containing 48 [Homo sapiens] |
| 80 | 11332 | 9606 | gi\|32528286 | acyl-CoA thioesterase 7 isoform hBACHd [Homo sapiens] |
| 81 | 6837 | 9606 | gi\|19557695 | mediator complex subunit 22 isoform b [Homo sapiens] |
| 82 | 10290 | 9606 | gi\|157785645 | SPEG complex locus [Homo sapiens] |
| 83 | 347735 | 9606 | gi\|71834872 | tumor differentially expressed 2-like [Homo sapiens] |
| 84 | 10480 | 9606 | gi\|23397429 | eukaryotic translation initiation factor 3, subunit M [Homo sapiens] |
| 85 | 1028 | 9606 | gi\|169790899 | cyclin-dependent kinase inhibitor 1C isoform b [Homo sapiens] |
| 86 | 3275 | 9606 | gi\|46255047 | HMT1 hnRNP methyltransferase-like 1 [Homo sapiens] |
| 87 | 10574 | 9606 | gi\|58331185 | chaperonin containing TCP1, subunit 7 isoform b [Homo sapiens] |
| 88 | 57176 | 9606 | gi\|55741845 | valyl-tRNA synthetase 2, mitochondrial [Homo sapiens] |
| 89 | 9080 | 9606 | gi\|11141861 | claudin 9 [Homo sapiens] |
| 90 | 782 | 9606 | gi\|40804472 | calcium channel, voltage-dependent, beta 1 subunit isoform 3 [Homo sapiens] |
| 91 | 7812 | 9606 | gi\|56117850 | upstream of NRAS isoform 2 [Homo sapiens] |
| 92 | 1974 | 9606 | gi\|83700235 | eukaryotic translation initiation factor 4A2 [Homo sapiens] |
| 93 | 51629 | 9606 | gi\|219555665 | solute carrier family 25, member 39 isoform a [Homo sapiens] |
| 94 | 63893 | 9606 | gi\|192449449 | ubiquitin-conjugating enzyme E2O [Homo sapiens] |
| 95 | 51706 | 9606 | gi\|49574502 | NAD(P)H:quinone oxidoreductase type 3, polypeptide A2 [Homo sapiens] |
| 96 | 83986 | 9606 | gi\|14042970 | integrin alpha FG-GAP repeat containing 3 [Homo sapiens] |
| 97 | 1327 | 9606 | gi\|4502981 | cytochrome c oxidase subunit IV isoform 1 precursor [Homo sapiens] |
| 98 | 27339 | 9606 | gi\|7657381 | PRP19/PSO4 pre-mRNA processing factor 19 homologue [Homo sapiens] |
| 99 | 871 | 9606 | gi\|32454741 | serine (or cysteine) proteinase inhibitor, clade H, member 1 precursor [Homo sapiens] |
| 100 | 3679 | 9606 | gi\|193785653 | unnamed protein product [Homo sapiens] |
| 101 | 4357 | 9606 | gi\|61835204 | mercaptopyruvate sulfurtransferase isoform 2 [Homo sapiens] |
| 102 | 51466 | 9606 | gi\|7706687 | Enah/Vasp-like [Homo sapiens] |
| 103 | 2266 | 9606 | gi\|70906437 | fibrinogen, gamma chain isoform gamma-A precursor [Homo sapiens] |
| 104 | 7695 | 9606 | gi\|4507987 | zinc finger protein 136 [Homo sapiens] |
| 105 | 4043 | 9606 | gi\|4505021 | low density lipoprotein receptor-related protein associated protein 1 precursor [Homo sapiens] |
| 106 | 8321 | 9606 | gi\|4503825 | frizzled 1 [Homo sapiens] |
| 107 | 10539 | 9606 | gi\|95113651 | glutaredoxin 3 [Homo sapiens] |
| 108 | 79639 | 9606 | gi\|42734434 | transmembrane protein 53 [Homo sapiens] |
| 109 | 51117 | 9606 | gi\|166795285 | coenzyme Q4 homologue [Homo sapiens] |
| 110 | 10540 | 9606 | gi\|5453629 | dynactin 2 [Homo sapiens] |
| 111 | 7710 | 9606 | gi\|145977222 | zinc finger protein 154 [Homo sapiens] |
| 112 | 1778 | 9606 | gi\|33350932 | cytoplasmic dynein 1 heavy chain 1 [Homo sapiens] |
| 113 | 6155 | 9606 | gi\|4506623 | ribosomal protein L27 [Homo sapiens] |
| 114 | 8936 | 9606 | gi\|4507913 | Wiskott-Aldrich syndrome protein family member 1 [Homo sapiens] |
| 115 | 1211 | 9606 | gi\|115527060 | clathrin, light polypeptide A isoform c [Homo sapiens] |
| 116 | 6217 | 9606 | gi\|4506691 | ribosomal protein S16 [Homo sapiens] |
| 117 | 10991 | 9606 | gi\|5870893 | solute carrier family 38, member 3 [Homo sapiens] |
| 118 | 1466 | 9606 | gi\|4503101 | cysteine and glycine-rich protein 2 [Homo sapiens] |
| 119 | 5447 | 9606 | gi\|127139033 | cytochrome P450 reductase [Homo sapiens] |
| 120 | 23070 | 9606 | gi\|24307983 | FtsJ methyltransferase domain containing 2 [Homo sapiens] |
| 121 | 4627 | 9606 | gi\|12667788 | myosin, heavy polypeptide 9, non-muscle [Homo sapiens] |
| 122 | 6165 | 9606 | gi\|16117791 | ribosomal protein L35a [Homo sapiens] |
| 123 | 3945 | 9606 | gi\|4557032 | L-lactate dehydrogenase B [Homo sapiens] |
| 124 | 84707 | 9606 | gi\|14249132 | brain expressed X-linked 2 [Homo sapiens] |
| 125 | 2778 | 9606 | gi\|117938765 | GNAS complex locus isoform g [Homo sapiens] |
| 126 | 10472 | 9606 | gi\|19923354 | zinc finger protein 238 isoform 2 [Homo sapiens] |
| 128 | 53407 | 9606 | gi\|8394376 | syntaxin 18 [Homo sapiens] |
| 129 | 2664 | 9606 | gi\|4503971 | GDP dissociation inhibitor 1 [Homo sapiens] |
| 130 | 147015 | 9606 | gi\|146231950 | dehydrogenase/reductase (SDR family) member 13 [Homo sapiens] |
| 131 | 3150 | 9606 | gi\|48255933 | high-mobility group nucleosome binding domain 1, isoform CRA_a [Homo sapiens] |
| 132 | 4023 | 9606 | gi\|4557727 | lipoprotein lipase precursor [Homo sapiens] |
| 133 | 79002 | 9606 | gi\|13128992 | hypothetical protein MGC2803 [Homo sapiens] |
| 134 | 10383 | 9606 | gi\|5174735 | tubulin, beta, 2 [Homo sapiens] |
| 135 | 57649 | 9606 | gi\|75677357 | PHD finger protein 12 isoform 1 [Homo sapiens] |
| 136 | 5214 | 9606 | gi\|11321601 | phosphofructokinase, platelet [Homo sapiens] |
| 137 | 7102 | 9606 | gi\|21265104 | tetraspanin 7 [Homo sapiens] |
| 138 | 322 | 9606 | gi\|4502131 | amyloid beta A4 precursor protein-binding, family B, member 1 isoform E9 [Homo sapiens] |
| 139 | 27344 | 9606 | gi\|7019519 | proprotein convertase subtilisin/kexin type 1 inhibitor precursor [Homo sapiens] |
| 140 | 3799 | 9606 | gi\|4758648 | kinesin family member 5B [Homo sapiens] |
| 141 | 23406 | 9606 | gi\|21624607 | coactosin-like 1 [Homo sapiens] |
| 142 | 8405 | 9606 | gi\|4507183 | speckle-type POZ protein [Homo sapiens] |
| 143 | 51655 | 9606 | gi\|7706359 | RAS, dexamethasone-induced 1 [Homo sapiens] |
| 144 | 146909 | 9606 | gi\|122937289 | kinesin family member 18B [Homo sapiens] |
| 145 | 22883 | 9606 | gi\|57242755 | calsyntenin 1 isoform 2 [Homo sapiens] |
| 146 | 64951 | 9606 | gi\|15721937 | mitochondrial ribosomal protein S24 [Homo sapiens] |
| 147 | 51147 | 9606 | gi\|189083821 | inhibitor of growth family, member 4 isoform 9 [Homo sapiens] |
| 148 | 55079 | 9606 | gi\|157388917 | FEZ family zinc finger 2 [Homo sapiens] |
| 149 | 51529 | 9606 | gi\|18777675 | APC11 anaphase promoting complex subunit 11 isoform 2 [Homo sapiens] |
| 150 | 10376 | 9606 | gi\|57013276 | tubulin, alpha, ubiquitous [Homo sapiens] |
| 151 | 5223 | 9606 | gi\|4505753 | phosphoglycerate mutase 1 (brain) [Homo sapiens] |
| 152 | 3312 | 9606 | gi\|16041670 | Unknown (protein for IMAGE: 3906958) [Homo sapiens] |
| 153 | 147007 | 9606 | gi\|22748979 | transmembrane protein 199 [Homo sapiens] |
| 154 | 6861 | 9606 | gi\|92859638 | synaptotagmin V [Homo sapiens] |
| 155 | 6144 | 9606 | gi\|18104948 | ribosomal protein L21 [Homo sapiens] |
| 156 | 6129 | 9606 | gi\|15431301 | ribosomal protein L7 [Homo sapiens] |
| 157 | 51510 | 9606 | gi\|189409150 | chromatin modifying protein 5 [Homo sapiens] |
| 158 | 3925 | 9606 | gi\|5031851 | stathmin 1 [Homo sapiens] |
| 159 | 6125 | 9606 | gi\|14591909 | ribosomal protein L5 [Homo sapiens] |
| 160 | 4904 | 9606 | gi\|34098946 | nuclease sensitive element binding protein 1 [Homo sapiens] |
| 161 | 2495 | 9606 | gi\|56682959 | ferritin, heavy polypeptide 1 [Homo sapiens] |
| 162 | 4637 | 9606 | gi\|17986258 | myosin, light chain 6, alkali, smooth muscle and non-muscle isoform 1 [Homo sapiens] |
| 163 | 1953 | 9606 | gi\|110347457 | EGF-like-domain, multiple 3 [Homo sapiens] |
| 164 | 4926 | 9606 | gi\|71361682 | nuclear mitotic apparatus protein 1 [Homo sapiens] |
| 165 | 56654 | 9606 | gi\|9050060 | NPDC-1 [Homo sapiens] |
| 166 | 293 | 9606 | gi\|156071462 | solute carrier family 25, member A6 [Homo sapiens] |
| 167 | 8772 | 9606 | gi\|4505229 | Fas-associated via death domain [Homo sapiens] |
| 168 | 10101 | 9606 | gi\|6912540 | nucleotide binding protein 2 (MinD homolog, E. coli) [Homo sapiens] |
| 169 | 9230 | 9606 | gi\|190358517 | RAB11B, member RAS oncogene family [Homo sapiens] |
| 170 | 8775 | 9606 | gi\|47933379 | N-ethylmaleimide-sensitive factor attachment protein, alpha [Homo sapiens] |
| 171 | 7280 | 9606 | gi\|4507729 | tubulin, beta 2 [Homo sapiens] |
| 172 | 2131 | 9606 | gi\|46370066 | exostosin 1 [Homo sapiens] |
| 173 | 1915 | 9606 | gi\|4503471 | eukaryotic translation elongation factor 1 alpha 1 [Homo sapiens] |
| 174 | 79004 | 9606 | gi\|148596996 | CUE domain containing 2 [Homo sapiens] |
| 175 | 7846 | 9606 | gi\|17986283 | tubulin, alpha 1a [Homo sapiens] |
| 176 | 4150 | 9606 | gi\|110347461 | MYC-associated zinc finger protein isoform 1 [Homo sapiens] |

1-16. (canceled)

17. A method for identifying specific marker sequences for diagnosis of prostate cancer and/or for prognosis in prostate cancer, comprising the following steps:
a) selecting patients with prostate cancer and high inflammation values and/or patients with prostate cancer and low inflammation values,
b) determining the interaction of a sample from the selected patients with marker sequences to be tested, wherein the marker sequences to be tested are placed on a solid support,
c) selecting marker sequences that demonstrate an interaction, and
d) determining whether the selected marker sequences distinguish between progressive and non-progressive prostate cancer.

18. The method according to claim 17, wherein the selected marker sequences distinguish between progressive and non-progressive prostate cancer with high inflammation values.

19. The method according to claim 17, wherein the selected patients belong to the same population group.

20. The method according to claim 17, wherein the sample from the selected patients is a bodily fluid or a tissue sample, in particular blood, whole blood, blood plasma, blood serum, patient serum, urine, cerebrospinal fluid, or synovial fluid.

21. The method according to claim 17, wherein the marker sequences to be tested are selected from the group comprising SEQ ID No. 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

22. The method according to claim 17, wherein the marker sequences to be tested are presented on a protein microarray.

23. Use of one or more specific marker sequences attainable by a method according to claim 17 for diagnosis of prostate cancer, preferably for diagnosis of prostate carcinoma.

24. Use of one or more specific marker sequences attainable by a method according to claim 17 for prognosis in prostate cancer and/or for stratification, in particular for risk stratification or for therapy control in prostate cancer.

25. Use of SPOP and/or partial sequences and/or homologues of SPOP and/or of STX18 and/or partial sequences and/or homologues of STX18 and/or of SPAST and/or partial sequences and/or homologues of SPAST for diagnosis of prostate cancer and/or for prognosis in prostate cancer and/or for stratification in prostate cancer.

26. An arrangement of specific marker sequences for diagnosis of prostate cancer and/or prognosis in prostate cancer and/or for stratification in prostate cancer, comprising one or more specific marker sequences obtainable by a method according to claim 17.

27. The arrangement according to claim 26, wherein the specific marker sequences are selected from the group comprising SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

28. The arrangement according to claim 26, wherein the specific marker sequences are selected from SPOP and/or partial sequences of SPOP and/or homologues of SPOP and/or STX18 and/or partial sequences of STX18 and/or homologues of STX18 and/or SPAST and/or partial sequences of SPAST and/or homologues of SPAST.

29. An assay or protein microarray comprising an arrangement of specific marker sequences according to claim 26 on a solid support and optionally further additives and excipients.

30. Use of an arrangement according to claim 26 for the identification and characterisation of a substance for prostate cancer, in particular a prostate carcinoma-containing agent for the detection of binding success, wherein a) the arrangement or the assay is brought into contact with at least one substance to be tested, and b) binding success is detected.

31. A diagnostic agent for diagnosis of prostate cancer and/or prognosis in prostate cancer, comprising an arrangement according to claim 26 and/or selected from the group of specific marker sequences SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

32. A diagnostic agent for diagnosis of prostate cancer and/or prognosis in prostate cancer, comprising one or more specific marker sequences obtainable by a method according to claim 17 and/or selected from the group of specific marker sequences SEQ ID No 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.

33. A kit for diagnosis or prognosis or stratification of prostate cancer diseases containing one or more specific marker sequences obtainable by a method according to claim 17 and/or one or more of the marker sequences selected from the group comprising SEQ ID No. 1-176 (proteins), SEQ ID No. 177-352 (DNA clone sequences) and SEQ ID No. 353-528 (related RNA sequences), partial sequences of SEQ ID No. 1-528 with at least 90%, preferably at least 95%, of the length of SEQ ID No. 1-528, and homologues of SEQ ID No. 1-528 with an identity of at least 95%, preferably at least 98% or more, to the corresponding marker sequences, and partial sequences of the homologues of SEQ ID No. 1-528 with at least 90%, preferably at least 95%, of the length of SEQ ID No. 1-528.
